(19) United States
(12) Patent Application Publication
(10) Pub. No.: US 2018/0194804 A1
(43) Pub. Date: Jul. 12, 2018

(54) SIMPLIFIED AND SCALABLE METHOD FOR SYNTHESIS OF 2,6-BIS(METHIONYL)- 1,4-DIKETOPIPERAZINE

(71) Applicant: Evonik Dequssa GmbH, Essen (DE)

(72) Inventors: Sascha BRAUNE, Bickenbach (DE); Daniel ROST, Mannheim (DE); Juergen BILZ, Freigericht (DE); Thomas HAEUSSNER, Eppertshausen (DE); Hans Joachim Hasselbach, Gelnhausen (DE); Christoph KOBLER, Alzenau (DE)

(73) Assignee: Evonik Degussa GmbH, Essen (DE)

(21) Appl. No.: 15/741,846

(22) PCT Filed: Jun. 28, 2016
(86) PCT No.: PCT/EP2016/064982

§ 371 Date: Jan. 4, 2018

(30) Foreign Application Priority Data
Jul. 9, 2015 (EP) 15176058.4

(51) Int. Cl. C07K 5/12

(52) U.S. Cl. CPC C07K 5/12

(57) ABSTRACT

The invention relates to a process for preparing 2,6-bis(methionyl)-1,4-dketopiperazine, comprising the following steps:
a) heating a reaction mixture comprising methionine and polar protic solvent at a temperature of less than 170.0° C., while passing an inert gas stream over or through the reaction mixture;
b) obtaining 2,6-bis(methionyl)-1,4-diketopiperazine.

The invention further relates to a process for isolating 2,6-bis(methionyl)-1,4-diketopiperazine from a reaction mixture, comprising the following steps:
A1) diluting the reaction mixture comprising 2,6-bis(methionyl)-1,4-diketopiperazine with a polar solvent and water, with crystallization of 2,6-bis(methionyl)-1,4-diketopiperazine; or
A2) diluting the reaction mixture comprising 2,6-bis(methionyl-1,4-diketopiperazine only with water without prior dilution with an organic solvent with or without pressure retention, with crystallization of 2,6-bis(mehionyl)-1,4-diketopiperazine; or
A3) cooling the reaction mixture down or allowing it to cool down without addition of substances until a temperature, especially of 135.0° C. to less than 145.0° C., preferably of 138.0° C. to 142.0° C., is reached, at which the diketopiperazine starts to crystallize, and finally diluting it and hence cooling it by addition of water, with crystallization of 2,6-bis(methionyl)-1,4-dketopiperazine;
B) removing the crystalized 2,6-bis(methionyl)-1,4-dketopiperazine obtained in A1) or A2) or A3) in solid form and washing it with a solvent, especially ethylene glycol, acetone or methanol, and finally washing with water.

More particularly, the present invention relates to a combination of a process for preparing 2,6-bis(methionyl)-1,4-diketopiperazine and a process for isolating 2,6-bis(methionyl)-1,4-diketopiperazine from a reaction mixture.

### INTRODUCTION

The present invention relates to novel chemical syntheses of 2,6-bis(methionyl)-1,4-diketopiperazine.

### PRIOR ART

There are various established prior art processes for preparation of 2,6-bis(methionyl)-1,4-dketopiperazine (met-DKP).

For instance, the U.S. Pat. No. 3,980,653 discloses a process for preparing 3,6-bis-(2-methylmercaptoethyl)-2,5-piperazinedione (methionine diketopiperazine) by reaction of methionine hydantoin with methionine at 160° C. and a pressure of initially 9.5 bar. The corresponding diketopiperazine was isolated by crystallization.

WO 2010/043558 discloses processes for preparing methionine diketopiperazine by conversion of N-carbamoylmethionine, N-carbamoylmethioninamide, methionylhydantoin, methioninamide, 3-(methylmercapto)propionaldehyde cyanohydrin, 3-(methylmercapto)propionaldehyde or methioninenitrile.

In addition, the literature describes processes for synthesis of DKP by direct dimerizatiorVcondensation of two amino acids in ethylene glycol or glycerol as solvents, wherein the processes are conducted at temperatures of 170-175° C. or higher (H. R Bentley et al. Pr. Roy. Soc. <B>1951, 138, 265; Sannié, Bull. Soc. Chim. 1942, 9(5), 487. Maillard, Compt. rend. 153, 1078 (1911): Ann. chim. et phys. [9]1, 521 (1914); 2, 210 (1914); 4, 225 (1915); Balbiano, Atti accad. Lincei, 23, I, 893 (1914), ibid., 24, I, 822, 936 (1915)). A disadvantage is that many impurities are formed at these high temperatures when using methionine or methionine analogues and have to be depleted by a complex workup which is impracticable on the production scale.

Alternatives described in the prior art are processes which increase the reaction rate by use, for example, of phosgene (A. D. Borthwick Chem. Rev. 2012, 112, 3641; A. Gonzalez et al. Tetrahedron Asymmetry 1995, 6, 1357; V. A. Basiuk et al. Synthesis 1992, 5, 449; V. Santagada et al. Tetrahedron Lett. 2003, 44, 1145; M. Jainta et al. Eur. J. Org. Chem. 2008, 32, 5418). However, there is no interest in adding further extraneous and possibly highly toxic components for industrial production with corresponding cost pressure.

Methionine is preferable as a starting material from a production point of view over other activated compounds, for example methionine methyl ester, since methionine is easily obtainable commercially and the activation entails an additional process step. The synthesis of 2,5-diketopiperazine proceeding from methionine methyl ester is already disclosed in publication DE 2 261 926 from 1972. It is disclosed therein that heating of the isopropyl ester of methionine forms 3,6-bis[2-(methylthio)ethy]-2,5-piperazinedione (methionine diketopiperazine, DKP). The publication by Baker, D. H. et al. (Journal of Nutrition, volume 114, no. 2, 1984, pages 292-297) also relates to this process for preparing diketopiperazine. However, the use of methionine isopropyl ester as starting material is too costly and therefore uneconomic.

Various very complex descriptions for the workup of the product mixture which are actually practicable only for the laboratory scale can be found in various sources referring to the publication by Sannié from 1942. The following publications describe the synthesis of other DKP derivatives: Arthur Vogel, Textbook of practical organic chemistry (4. ed.rev.); Longman, 1981, New York, 909; H. F. Schott J. Org. Chem. 1947, 12, 490.

### Problem Addressed by the Invention

The problem addressed by the present invention was that of providing a novel, simpler and less expensive process implementable on the production scale for preparation of 2,6-bis(methionyl)-1,4-diketopiperazine.

### DESCRIPTION OF THE INVENTION

In a first aspect of the present invention, a process for preparing 2,6-bis(methionyl)-1,4-diketopiperazine is provided, comprising the following steps:

a) heating a reaction mixture comprising methionine and polar protic solvent at a temperature of less than 170.0° C., while passing an inert gas stream over or through the reaction mixture;

b) obtaining 2,6-bis(methionyl)-1,4-diketopiperazine.

According to the present invention, the reaction temperature in the conversion of methionine to DKP is below 170.0° C. In contrast, the processes described in the prior art are conducted at temperatures of 170-175° C. or higher. Many impurities are formed at these high temperatures when using methionine and have to be depleted by a complex workup which is impracticable on the production scale. The milder reaction conditions combined with comparable reaction times in the process according to the present invention result in formation of a much lower level of by-products, which is apparent from the colour of the reaction mixture. Thus, a particular advantage of the process according to the invention is the purity of the diketopiperazine obtained. This allows easy, rapid and hence inexpensive purification from the reaction mixture by crystallization.

In further preferred processes, the temperature in step a) is not more than 169.5° C., preferably not more than 169.0° C., further preferably not more than 168.0° C., even further preferably not more than 166.0° C. and especially preferably 165.0° C.

It is especially preferable that the temperature in step a) is from 145.0° C. to 169.5° C., preferably from 145.0° C. to 169.0° C., further preferably from 145.0° C. to 168.0° C., even further preferably from 145.0° C. to 166.0° C. and especially preferably from 145.0° C. to 165.0° C.

It is further preferable that the temperature in step a) is from 155.0° C. to 169.5° C., preferably from 155.0° C. to 169.0° C., further preferably from 155.0° C. to 168.0° C., even further preferably from 155.0° C. to 166.0° C. and especially preferably from 155.0° C. to 165.0° C.

In a further particularly preferred process, the temperature in step a) is from 160.0° C. to 169.5° C., preferably from 160.0° C. to 169.0° C., further preferably from 160.0° C. to 168.0° C., even further preferably from 160.0° C. to 166.0° C. and especially preferably from 160.0° C. to 165.0° C.

With regard to the effects of reaction temperature, more exact studies were conducted. For instance, it was found that the speed of the reaction would be optimal at a temperature of about 175° C. However, as already elucidated above, at temperatures above the temperatures employed according to the invention, the accompanying polymerization and decomposition reactions are significant. These form unwanted impurities which have to be removed with a higher level of complexity and hence higher costs. In addition, it was found that vigorous decomposition reactions can occur even at temperatures of 180° C.−210° C., which would be prohibitive to an industrial process. The process according to the invention allows a minimum margin from the initiation temperature of the decomposition to be observed. The process according to the present invention thus works with milder reaction conditions, such that a much lower level of by-products is formed as well, but still with acceptable conversion times.

In a preferred process, the initial concentration of the methionine in the reaction mixture is 20% by weight to 60% by weight, more preferably 30% by weight to 50% by weight. In this case, the initial mixing ratio (w/w) of methionine to polar protic solvent in the reaction mixture is preferably from 1:4 to 4:1, especially from 1:4 to 1.5:1. Compliance with these preferred parameters has a positive effect on the crystallization of the product described below, which is induced by dilution of the reaction solution after the reaction.

Above an initial concentration of the methionine in the reaction mixture of 60% by weight, there is a distinct deterioration in the kinetics of the reaction. This is caused by the process regime, in which DKP is formed and crystallized in the same reactor. At higher concentrations of methionine, the volume during the reaction is lower and the effectiveness of passing the inert gas over is poorer. In an alternative process, the initial concentration of methionine may also be above 60% by weight, with formation and crystallization of DKP in separate reactors.

In a preferred process, the reaction mixture used is a mixture consisting essentially of a polar protic solvent and methionine.

In a further preferred process, the polar protic solvent is a polyhydric alcohol, especially a dihydric or trihydric alcohol. Further preferably, the polar protic solvent is a polyhydric alcohol, especially a dihydric or trihydric alcohol, having 24 carbon atoms, especially 2 or 3 carbon atoms. Further preferably still, the polar protic solvent is selected from the group consisting of glycols, ethylene glycol, propane-1,2-diol, propane-1,3-diol, glycerol and butanediol, more preferably ethylene glycol. In addition, it is possible to use mixtures of polyhydric alcohols as polar protic solvent, especially a mixture of ethylene glycol and glycerol. In addition, solvents used may be mixtures of one or more polyhydric alcohols, especially dihydric or trihydric alcohols, having 2-4 carbon atoms, especially 2 or 3 carbon atoms, with another organic solvent having a boiling point of more than 180° C.

In a further preferred process, the polar protic solvent used is at least partly recycled. In addition, it is preferable that the polar protic solvent used is recycled to an extent of at least 80%, preferably to an extent of at least 85%, especially preferably to an extent of at least 90% and very especially preferably to an extent of at least 91%. The high recovery rate of the solvent and the reuse thereof decreases costs.

The polar protic solvent, especially the above-described polyhydric alcohol, serves as activating reagent for the carboxyl group of a methionine molecule, with possible intermediate formation of the corresponding ester, which is depleted under the given reaction conditions by reaction with the amino group of a second methionine molecule. The subsequent cydization to give the corresponding diketopiperazine proceeds analogously. Since the intramolecular reaction to give a six-membered ring is kinetically favoured under these reaction conditions, the reaction cannot be stopped at the dipeptide intermediate. Without being bound to a theory, it can be assumed that a methionine dipeptide is formed at first and then is cydized directly or after a further activation to give DKP. At the start of the reaction, a suspension is present in the reaction mixture, from which a solution forms with increasing conversion. The DKP crystallizes out of the solution after the reaction has ended, as described further down.

It is additionally preferable that step a) of the process according to the invention is conducted for 1 to 48 h, preferably for 1 to 24 h, further preferably for 1 to 15 h and more preferably for 4 to 7 h.

According to the invention, an inert gas stream is passed over or through the reaction mixture.

In one process variant of the process according to the invention, the inert gas stream is passed over (not through) the reaction mixture. The measure that an inert gas stream is passed “over the reaction mixture” means that this inert gas stream is conducted above the liquid level of the reaction mixture. This means that the inert gas stream is not introduced into the reaction mixture or conducted therethrough.

In an alternative process variant of the process according to the invention, the inert gas stream is passed through the reaction mixture. In order to avoid cooling of the reaction mixture as a result of the passage of the inert gas, or in order to keep the reaction mixture at the desired temperature or to bring it to this temperature, the inert gas on introduction into the reaction mixture has a temperature of at least 100.0° C., preferably at least 120.0° C., further preferably at least 145.0° C., even further preferably at least 155.0° C. and more preferably at least 160.0° C.

This measure—passing an inert gas stream over the reaction mixture or above the reaction mixture or through the reaction mixture—serves to remove the water which is released in the double condensation from the equilibrium by means of zeotropic distilation, in order to achieve acceptable reaction times. Without the application of an inert gas stream, the reaction times would extend unacceptably to dimensions of days. With regard to the inert gas stream, it is preferable that the inert gas used is nitrogen, carbon dioxide, oxygen-depleted air (lean air), combustion gases or noble gases such as argon or helium. In a particularly preferred process, nitrogen is used as inert gas.

A particular advantage of the process according to the invention is the purity of the diketopiperazine obtained. This allows easy and rapid purification from the reaction mixture by direct crystallization. Thus, in the process according to the invention, by contrast with the prior art processes, there is no longer any need to conduct an extraction prior to the crystalization, in order to deplete impurities. In a preferred embodiment of the process according to the invention, the reaction mixture, after the end of the reaction or in order to end the reaction, is diluted with a polar solvent and water and cooled down to below 100.0° C. This crystalizes the 2,6-bis(methionyl)-1,4-dietopiperazine. The crystalized 2,6-bis(methionyl)-1,4-dikeopiperazine can then be removed in solid form and washed to whiteness with ethylene glycol, acetone or methanol and finally freed of organic solvent residues with water. Typically, a wool-white product is obtained. The solid can be removed here in an advantageous manner by means of a pressure filter press, centrifuge, belt filter or a comparable sold/liquid separation apparatus. The removal of solids is advantageously and therefore preferably accomplished at room temperature.

In the process according to the invention, the polar solvent used to dilute the reaction mixture is preferably the same solvent which has been used in the reaction mixture in the reaction.

Preferably, the polar solvent is a polar protic solvent, more preferably a polyhydric alcohol, especially a dihydric or trihydric alcohol. Further preferably, the polar solvent is a polyhydric alcohol having 2-4 carbon atoms, especially 2 or 3 carbon atoms. Further preferably, the polar solvent is selected from the group consisting of the glycols, ethylene glycol, propane-1,2-diol, propane-1,3-diol, glycerol and butanediol, preferably ethylene glycol. Furthermore, it is possible to use mixtures of polyhydric alcohols as polar solvent, especially a mixture of ethylene glycol and glycerol.

In a further preferred process of the present invention, the reaction mixture, after the reaction or to end the reaction, is diluted with a polar solvent, more preferably with ethylene glycol, and then diluted with water and at the same time cooled down to below 100.0° C., preferably to 95.0° C. to 98.0° C., with crystallization of the 2,6-bis(methionyl)-1,4-diketopiperazine.

More preferably, the reaction mixture is diluted with a polar solvent, most preferably with ethylene glycol, and at the same time cooled down to 125.0° C. to 135.0° C., and then diluted with water and at the same time cooled down to below 100.0° C., preferably to 95.0° C. to 98.0° C., with crystalization of the 2,6-bis(methionyl)-1,4-dikeopiperazine.

In the course of dilution, the ratio (w/w) of the polar solvent added to the reaction mixture, especially ethylene glycol, to the water added is preferably 1:4 to 4:1, especially 4:3 to 3:1. With regard to the dilution step, it is preferable that the temperature of the reaction mixture prior to the dilution or on commencement of the dilution with the polar solvent is at least 145.0° C., preferably at least 150.0° C., further preferably at least 155.0° C., even further preferably at least 160.0° C.

The dilution of the reaction mixture results in the cooing thereof. This also ends the reaction. Depending on the starting concentration, a sufficient amount of solvent can be added that a solution is still just present or the first crystals precipitate out. The crystallization of the 2,6-bis(methionyl)-1,4-diketopiperazine is initiated by the change in the polarity on addition of the water. The dilution with water assures the formation of crystalline 2,6-bis(methionyl)-1,4-diketopiperazine having the crystal structure suitable for further processing. The crystalline 2,6-bis(methionyl)-1,4-diketopiperazine can then be removed in solid form and washed to whiteness with ethylene glycol, acetone or methanol and finally freed of organic solvent residues with water, which is advantageously and therefore preferably accomplished at room temperature.

In an alternative embodiment, the reaction mixture, after the reaction has ended or in order to end the reaction, is cooled down by diluting solely with water without prior dilution with an organic solvent, with crystallization of 2,6-bis(methionyl)-1,4-diketopiperazine. The addition of the water can be adjusted such that the reaction mixture cools down especially to 135.0° C. or lower, preferably to 120.0° C. or lower, further preferably to 110° C. or lower and even further preferably to below 100.0° C.

In this case, the addition of the water for dilution and cooing of the reaction mixture can be effected with or without pressure retention.

If the dilution and cooling of the reaction mixture is effected solely by addition of water without prior dilution with an organic solvent with employment of pressure retention, the addition of the water is adjusted in such a way that the reaction mixture is cooled down especially to temperatures above 100.0° C. to 135.0° C., preferably above 100.0° C. to 120.0° C., more preferably above 100.0° C. to 110.0° C., and further cooling to below 100.0° C. is effected by decompression to atmospheric pressure. In a preferred embodiment, the addition of the water for dilution and cooling of the reaction mixture is adjusted in such a way that a gauge pressure of 0.0 to 7.0 bar, preferably a gauge pressure of 0.0 to 3.0 bar and more preferably a gauge pressure of 0.0 to 2.0 bar is applied.

The particular advantage of the measure of pressure retention on addition of the water for dilution and for cooling is that it is possible in the case of this procedure to use less water for the cooling of the reaction mixture. The reason for this is that the reaction mixture is cooled down further on decompression to atmospheric pressure. At the same time, this reduces the residual amount of water in the mother liquor to be distilled off. According to this process variant, in which the reaction is followed by the addition of water to cool the reaction mixture down to a temperature below 100.0° C. with pressure retention (preferably to 7.0 bar, further preferably to 3.0 and even further preferably to 2.0 bar gauge), less water has to be added to cool the reaction mixture than when cooling is to be effected directly to a temperature below 100.0° C. In the case of pressure retention, it is necessary to add less water because, on decompression to atmospheric pressure, the reaction mixture is cooled down further to 100° C. or lower by the enthalpy of evaporation of the water. This draws off a portion of the water, which means that the water content in the mother liquor is then lower than in the first case. This leads to a lower level of complexity or less time spent in the redistillation of the mother liquor.

The crystalized 2,6-bis(methionyl)-1,4-dketopiperazine can then be removed in sold form and washed to whiteness with ethylene glycol, acetone or methanol and finally freed of organic solvent residues with water. In this embodiment, with regard to the dilution step, it is preferable that the temperature of the reaction mixture prior to the dilution or on commencement of the dilution with water is at least 145.0° C., preferably at least 150.0° C., further preferably at least 155.0° C., even further preferably at least 160.0° C.

In a further alternative embodiment, the reaction mixture, after the reaction has ended or in order to end the reaction, is cooled down or allowed to cool down without addition of substances until it reaches a temperature, especially of 135.0° C. to less than 145.0° C., preferably of 138.0° C. to 142.0° C., at which the diketopiperazine starts to crystallize, and finally diluted and cooled further by addition of water. This crystallizes the 2,6-bis(methionyl)-1,4-diketopiperazine, which can then be removed in solid form and washed to whiteness with ethylene glycol, acetone or methanol and finally freed of organic solvent residues with water.

The wash step conducted with water serves to deplete the polar solvent used for extraction beforehand. The solvents used for the washing of the product can likewise be distilled again and recycled. Therefore, in a further preferred process, the solvent used to wash the crystallized 2,6-bis(methionyl)-1,4-diketopiperazine is at least partly recycled. The reutilization rates of the solvent used for washing may be at least 90%, preferably at least 95%, more preferably at least 97%.

The sold obtained can be removed here in an advantageous manner by means of a pressure filter press, centrifuge, a belt filter or a comparable solid/liquid separation apparatus, which is advantageously and therefore preferably accomplished at room temperature.

Washing of the crystallized 2,6-bis(methionyl)-1,4-dketopiperazine can be effected using the solvent used, but preference is given to using a polar solvent different from that which is used in the reaction mixture. Preferably, the polar solvent used to wash the crystalized 2,66-bis(methionyl)-1,4-diketopiperazine is a polar solvent selected from the group consisting of nitriles, especially acetonitrile; cyclic and acyclic carboxylic esters, especially ethyl acetate; aromatic solvents, especially toluene; cyclic and acyclic carboxamides, especially dimethylformamide (DMF) and N-methyl-2-pyrrolidone (NMP); carbonic esters, especially dimethyl carbonate; cyclic and acyclic ethers, especially tetrahydrofuran (THF) and 2-methoxy-2-methylpropane (MTBE); ketones, especially acetone; sulphoxides, especially dimethyl sulphoxide (DMSO); amines, especially triethylamine; carboxylic acids, especially acetic acid; chlorinated hydrocarbons, especially dichloromethane and chloroform; alcohols, especially methanol, ethanol, n-propanol, isopropyl alcohol; preferably acetone or methanol.

### Second Aspect of the Invention

As already explained, a particular advantage of the present invention is the purity of the diketopiperazine obtained by the process. The purity of the product at the end of the reaction enables a simple and rapid purification from the reaction mixture by direct crystallization. As already described above, in a preferred embodiment of the process, the reaction mixture, after the reaction has ended or in order to end the reaction, is either diluted with a polar solvent and water and cooled down to below 100.0° C. or solely diluted with water without prior dilution with an organic solvent, or cooled down or allowed to cool down without addition of substances until a temperature is reached at which the diketopiperazine begins to crystallize, and finally diluted and cooled down by addition of water. This crystallizes the 2,6-bis(methionyl)-1,4-diketopiperazine, which can then be removed in solid form and washed to whiteness with ethylene glycol, acetone or methanol and finally freed of organic solvent residues with water.

Therefore, the present invention, in a second aspect, provides a process for isolating 2,6-bis(methionyl)-1,4-diketopiperazine from a reaction mixture, comprising the following steps:
A1) diluting the reaction mixture comprising 2,6-bis(methionyl)-1,4-diketopiperazine with a polar solvent and water, with crystalization of 2,6-bis(methionyl)-1,4-diketopiperazine; or
A2) diluting the reaction mixture comprising 2,6-bis(methionyl)-1,4-diketopiperazine only with water without prior dilution with an organic solvent with or without pressure retention, with crystallization of 2,6-bis(methionyl)-1,4-dketopiperazine; or
A3) cooling the reaction mixture down or allowing it to cool down without addition of substances until a temperature, especially of 135.0° C. to less than 145.0° C., preferably of 138.0° C. to 142.0° C., is reached, at which the diketopiperazine starts to crystallize, and finally diluting it and hence cooling it by addition of water, with crystallization of 2,6-bis(methionyl)-1,4-dketopiperazine;
B) removing the crystalized 2,6-bis(methionyl)-1,4-dketopiperazine obtained in A1) or A2) or A3) in solid form and washing it with a solvent, especially ethylene glycol, acetone
or methanol, and finally washing it with water.

The wash step conducted with water in step B) serves to deplete the polar solvent used for extraction beforehand, which allows a product of high purity to be obtained in a very simple and advantageous manner. The solvents used in the wash steps (step B)) can likewise be distilled again and recycled. Therefore, in a further preferred process, the solvent used to wash the crystallized 2,6-bis(methionyl)-1,4-diketopiperazine is at least partly recycled. The reutilization rates of the solvent used for washing may be at least 90%, preferably at least 95%, more preferably at least 97%.

Especially according to variant A1, a product of high purity is obtained in a simple manner and without additional complexity and also within relatively short residence times. Therefore, variant A1 is particularly advantageous.

The solid from step B) can be removed here in an advantageous manner by means of a pressure filter press, centrifuge, a belt filter or a comparable solid/liquid separation apparatus, which is advantageously and therefore preferably accomplished at room temperature.

In the process according to the second aspect of the invention, the reaction mixture, because of the reaction that has already taken place, consists essentially of 2,6-bis(methionyl)-1,4-diketopiperazine, the polar protic solvent originally used and unconverted methionine, and possibly water and by-products of the reaction.

In the process according to the second aspect of the invention, the polar solvent used to dilute the reaction mixture according to step A1) is preferably the same solvent which has been used in the reaction mixture for the reaction. Preferably, the polar solvent is a polar protic solvent, more preferably a polyhydric alcohol, especially a dihydric or trihydric alcohol. Further preferably, the polar solvent is a polyhydric alcohol having 2-4 carbon atoms, especially 2 or 3 carbon atoms. Further preferably, the polar solvent is selected from the group consisting of the glycols, ethylene glycol, propane-1,2-diol, propane-1,3-diol, glycerol and butanediol, preferably ethylene glycol. Furthermore, it is possible to use mixtures of polyhydric alcohols as polar solvent, especially a mixture of ethylene glycol and glycerol.

In a further preferred process of the second aspect of the invention, the reaction mixture in step A1) is diluted with a polar solvent, and then diluted with water and at the same time cooled down to below 100.0° C., preferably to 95.0° C. to 98.0° C., with crystallization of the 2,6-bis(methionyl)-1,4-diketopiperazine.

In a further very particularly preferred process of the second aspect of the invention, the reaction mixture in step A1) is diluted with ethylene glycol, and then diluted with water and at the same time cooled down to below 100.0° C., preferably to 95.0° C. to 98.0° C., with crystallization of the 2,6-bis(methionyl)-1,4-diketopiperazine.

In the process according to the second aspect of the invention, in the dilution, the ratio (w/w) of the polar solvent added to the reaction mixture, especially ethylene glycol, to the water added is preferably 1:4 to 4:1, especially 4:3 to 3:1.

In the process according to the second aspect of the invention, the temperature of the reaction mixture prior to the dilution or on commencement of the dilution according to step A1) is at least 145.0° C., preferably at least 150.0° C., further preferably at least 155.0° C., even further preferably at least 160.0° C.

According to process variant A2), the reaction mixture, after the reaction has ended or in order to end the reaction, is cooled down by diluting solely with water without prior dilution with an organic solvent, with crystallization of 2,6-bis(methionyl)-1,4-diketopiperazine. The addition of the water can be adjusted such that the reaction mixture cools down especially to 135.0° C. or lower, preferably to 120.0° C. or lower, further preferably to 110° C. or lower and even further preferably to below 100.0° C.

In step A2), the addition of the water for dilution and cooling of the reaction mixture can be effected with or without pressure retention.

If the dilution and cooling of the reaction mixture is effected solely by addition of water without prior dilution with an organic solvent with employment of pressure retention, the addition of the water is adjusted in such a way that the reaction mixture is cooled down especially to temperatures above 100.0° C. to 135.0° C., preferably above 100.0° C. to 120.0° C., more preferably above 100.0° C. to 110.0° C., and further cooling to below 100.0° C. is effected by decompression to atmospheric pressure. In a preferred embodiment, the addition of the water for dilution and cooling of the reaction mixture is adjusted in such a way that a gauge pressure of 0.0 to 7.0 bar, preferably a gauge pressure of 0.0 to 3.0 bar and more preferably a gauge pressure of 0.0 to 2.0 bar is applied.

The crystalized 2,6-bis(methionyl)-1,4-dketopiperazine can then be removed in solid form and washed to whiteness with ethylene glycol, acetone or methanol and finally freed of organic solvent residues with water. In this embodiment, with regard to the dilution step, it is preferable that the temperature of the reaction mixture prior to the dilution or on commencement of the dilution with water is at least 145.0° C., preferably at least 150.0° C., further preferably at least 155.0° C., even further preferably at least 160.0° C.

In step A3), the reaction mixture, after the reaction has ended or in order to end the reaction, is cooled down or allowed to cool down without addition of substances until it reaches a temperature, especially of 135.0° C. to less than 145.0° C., preferably of 138.0° C. to 142.0° C. at which the diketopiperazine starts to crystallize, and finally diluted and cooled further by addition of water. This crystallizes the 2,6-bis(methionyl)-1,4-diketopiperazine, which can then be removed in solid form and washed to whiteness with ethylene glycol, acetone or methanol and finally freed of organic solvent residues with water.

In the process according to the second aspect of the invention, the polar solvent used to wash the crystallized 2,6-bis(methionyl)-1,4-dketopiperazine according to step B) is a solvent different from that which is used in the reaction mixture. Preferably, the polar solvent used to wash the crystallized 2,6-bis(methionyl)-1,4-dketopiperazine according to step B) is a polar solvent selected from the group consisting of nitriles, especially acetonitrile; cyclic and acyclic carboxylic esters, especially ethyl acetate; aromatic solvents, especially toluene; cyclic and acyclic carboxamides, especially dimethylformamide (DMF) and N-methyl-2-pyrrolidone (NMP); carbonic esters, especially dimethyl carbonate; cyclic and acyclic ethers, especially tetrahydrofuran (THF) and 2-methoxy-2-methylpropane (MTBE); ketones, especially acetone; sulphoxides, especially dimethyl sulphoxide (DMSO); amines, especially triethylamine; carboxylic acids, especially acetic acid; chlorinated hydrocarbons, especially dichloromethane and chloroform; alcohols, especially methanol, ethanol, n-propanol, isopropyl alcohol; preferably acetone or methanol.

### Overall Process for Preparation and Isolation of 2,6-bis(methionyl)-1,4-diketopiperazine

As elucidated above, the present invention relates in a first aspect to a process for preparing 2,6-bis(methionyl)-1,4-diketopiperazine and provides in a second aspect a process for isolating 2,6-bis(methionyl)-1,4-diketopiperazine from a reaction mixture.

In addition, the present invention provides a process corresponding to a combination of the first and second aspects of the present invention. Therefore, the present invention especially relates to a process for preparing 2,6-bis(methionyl)-1,4-diketopiperazine having the formula (I)

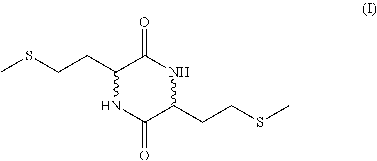

comprising the following steps:

a) heating a reaction mixture comprising methionine and a polar protic solvent at a temperature of less than 170.0° C., while passing an inert gas stream over or through the reaction mixture, preferably over the reaction mixture, in order to remove the water formed in the reaction from the reaction mixture;

b) obtaining 2,6-bis(methionyl)-1,4-diketopiperazine (formula (I));

c1) diluting the reaction mixture with polar solvent and water to crystallize 2,6-bis(methionyl)-1,4-diketopiperazine (formula (I)); or

c2) diluting the reaction mixture only with water without prior dilution with an organic solvent with or without pressure retention, to crystallize 2,6-bis(methionyl)-1,4-diketopiperazine (formula (I)); or

c3) cooling the reaction mixture down or allowing it to cool down without addition of substances until a temperature, especially of 135.0° C. to less than 145.0° C., preferably of 138.0° C. to 142.0° C. is reached, at which the diketopiperazine starts to crystallize, and finally diluting it and hence cooling it by addition of water, to crystallize 2,6-bis(methionyl)-1,4-diketopiperazine (formula (I)).

This process according to the combination of the first and second aspects of the present invention, in a corresponding manner, also includes the preferred embodiments, features, technical effects and advantages which have been described in relation to the first and second aspects, and to which reference is made here.

In the search for a process for preparing 2,6-bis(methionyl)-1,4-dketopiperazine (DKP) which does not employ an activated form of methionine, the inventors have become aware of the synthesis proceeding from methionine. As already elucidated above, the synthesis of DKP described in the literature is effected by direct dimerization/condensation of two amino acids in ethylene glycol or glycerol as solvents at temperatures of 170-175° C. or higher (H. R. Bentley et al. Pr. Roy. Soc. <B>191, 138, 265; Sannié, Bull. Soc. Chim. 1942, 9(5), 487. Maillard, Compt. rend. 153, 1078 (1911); Ann. chim. et phys. [9]1, 521 (1914); 2, 210 (1914); 4, 225 (1915); Balbiano, Atti accad. Lincei, 23, I, 893 (1914), ibid., 24, I, 822, 936 (1915)). However, at these high temperatures, many impurities are formed in the case of use of methionine as a result of the polymerization and decomposition reactions that likewise proceed. These unwanted impurities have to be depleted or removed by a complex workup which is impracticable on the production scale.

In the context of the present invention, it has been possible to simplify the workup in such a way that the process can even be used on the industrial scale. According to the present invention, the impurities that occur in the synthesis of DKP can be minimized. Two measures are essential to the invention:
i) The reaction temperature in the conversion of methionine to DKP in step a) is below 170.0° C. The temperatures are preferably not more than 169.5° C., not more than 169.0° C., not more than 168.0° C., not more than 166.0° C., not more than 165.0° C. More particularly, the following temperature ranges are particularly preferred: 160.0° C. to 169.5° C., 160.0° C. to 169.0° C., 160.0° C. to 168.0° C., 160.0° C. to 166.0° C.; very particular preference is given to the temperature range of 160.0° C. to 165.0° C.
ii) In addition, in step a) of the process according to the invention, an inert gas stream, especially a nitrogen stream, is passed over or through the reaction mixture, in order to remove the water which is released in the synthesis of the DKP from the equilibrium by zeotropic distillation.

The lower temperatures which are employed in the process according to the invention as compared with the processes described in the prior art lead to milder reaction conditions combined with comparable reaction times. This measure has a direct positive effect on the product, since a much lower level of by-products is formed here. The higher purity of the diketopiperazine obtained by the process according to the invention enables an easier, quicker and less expensive removal and purification from the reaction mixture by means of crystallization.

A further advantage of the process according to the invention is that the means of heating or the heating jacket in the reactors and apparatuses that exist in the prior art generally have a vacuum-stable design. Apparatuses having a vacuum-stable design can generally be operated even up to a pressure in the region of 6 bar. When these reactors and apparatuses are used and when a pressure in the region of 6 bar is employed in the steam-heated heating jacket or heating loop, the temperature of 160.0° C. to 165.0° C. which is particularly preferred and advantageous for the process according to the invention is achieved in the course of heating. Therefore, the process according to the invention can advantageously be conducted in existing multipurpose plants with existing apparatuses, which are typically designed for this pressure.

The process according to the invention is elucidated in detail by the example which follows, but this should not be regarded as being restrictive of the invention described here.

### EXAMPLE

### Example 1: DKP Synthesis

The synthesis of diketopiperazine was effected according to the following reaction scheme:

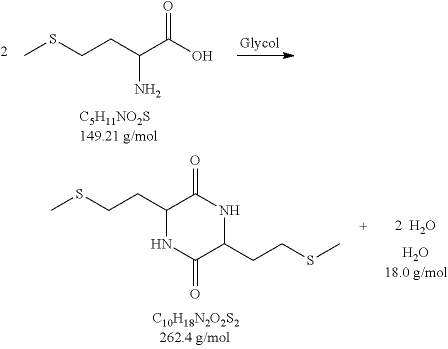

9.0 kg of ethylene glycol (d=1.11 g/ml) were initially charged in the 20 I reactor, and 6.0 kg of methionine (40.2 mol) were added while stirring. The beige suspension which had good stirrability was heated to 165.0° C. At the same time, a nitrogen stream (200 I (STP)/h) was passed over the surface to drive out the water formed. The offgases were odorous and toxic and were cleaned with a gas wash bottle filled with 15% hydrogen peroxide solution. After a reaction time of 4 hours at 165.0° C., a conversion of 90% was observed, and after a total of 6 hours, 95% conversion. A red-brown solution had formed, and 1.2 kg of water-ethylene glycol mixture were driven out.

For workup, a further 4.0 kg of ethylene glycol were added to the reaction mixture at 160.0° C. This cooled it down to 130.0° C. In the course of cooing to 130.0° C., DKP began to crystallize. As a result of metered addition of 3.0 kg of water, the reaction mixture cooled down to 95.0° C. (gentle foaming) and formed a yellow-brown suspension of good filterability. The mixture was cooled down very rapidly to room temperature.

The suspension was filtered with suction using a pressure filer press (MN 616 filter paper). Alternatively, it is possible to use a centrifuge. The wool-white filter cake (ø29 cm) was washed once with 13 I of acetone on the suction filter. The cake was not digested. Thereafter, washing was effected twice more with 10 I of water in order to reduce the ethylene glycol and acetone content. 5.33 kg of wool-white DKP (16.3 mol) having a DKP content of 80% were removed, corresponding to a yield of 80%.

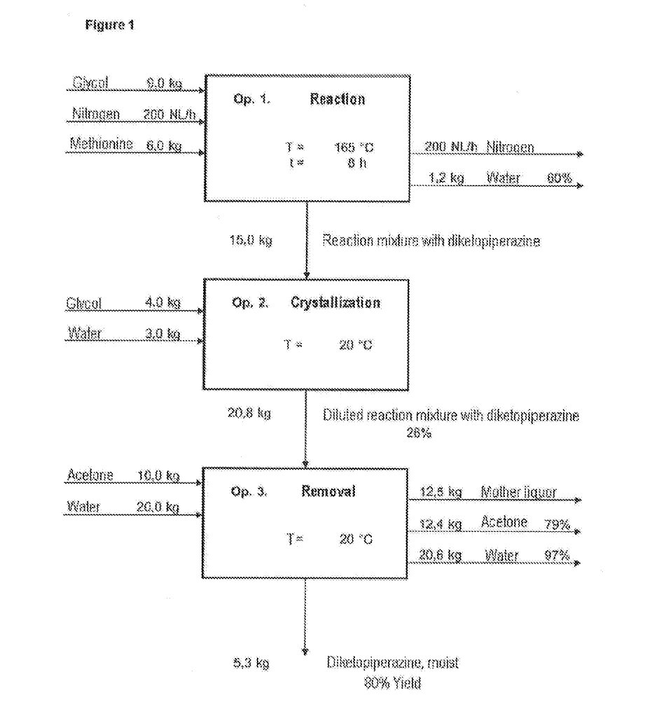

FIG. 1 shows the scheme for preparation, crystallization and purification of the diketopiperazine.

1: A process for preparing 2,6-bis(methionyl)-1,4-diketopiperazine, comprising:
a) heating a reaction mixture comprising methionine and polar protic solvent at a temperature of from 145.0° C. to 169.5° C., while passing an inert gas stream over or through the reaction mixture; and
b) obtaining 2,6-bis(methionyl)-1,4-diketopiperazine.

2: The process according to claim 1, wherein the temperature in a) is not more than 169.0° C.

3: The process according to claim 1, wherein the temperature in a) is from 145.0° C. to 168.0° C.

4: The process according to claim 1, wherein the temperature in a) is from 155.0° C. to 169.5° C.

5: The process according to claim 1, wherein the temperature in a) is from 160.0° C. to 169.5° C.

6: The process according to claim 1, wherein an initial concentration of methionine in the reaction mixture is 20% by weight to 60% by weight.

7: The process according to claim 1, wherein an initial mixing ratio (w/w) of methionine to polar protic solvent in the reaction mixture is from 1:4 to 4:1.

8: The process according to claim 1, wherein the polar protic solvent is a polyhydric alcohol.

9: The process according to claim 1, wherein the polar protic solvent used is recycled at least partly, to an extent of at least 80%.

10: The process according to claim 1, wherein the inert gas stream is passed over the reaction mixture, in order to remove the water formed in the reaction from the reaction mixture.

11: The process according to claim 1, wherein the inert gas stream is passed through the reaction mixture, in order to remove the water formed in the reaction from the reaction mixture, where the inert gas on introduction into the reaction mixture has a temperature of at least 100.0° C.

12: The process according to claim 10, wherein the inert gas used is nitrogen, carbon dioxide, oxygen-depleted air (lean air), combustion gases or noble gases.

13: The process according to claim 1, wherein
c1) the reaction mixture is cooled down to below 100.0° C. by diluting with a polar solvent and water, with crystallization of 2,6-bis(methionyl)-1,4-diketopiperazine; or
c2) the reaction mixture is cooled down with or without pressure retention solely by diluting with water without prior dilution with an organic solvent, with crystallization of 2,6-bis(methionyl)-1,4-diketopiperazine; or
c3) the reaction mixture is cooled down or allowed to cool down without addition of substances until it reaches a temperature at which the diketopiperazine starts to crystallize, and finally diluted and cooled further by addition of water, with crystallization of 2,6-bis(methionyl)-1,4-diketopiperazine.

14: The process according to claim 13, wherein, c1) is preformed, and in c1), the reaction mixture, after the reaction or to end the reaction, is diluted with a polar solvent, and then diluted with water and at the same time cooled down to below 100.0° C., with crystallization of the 2,6-bis(methionyl)-1,4-diketopiperazine.

15: The process according to claim 13, wherein the crystallized 2,6-bis(methionyl)-1,4-diketopiperazine is removed in solid form, washed with ethylene glycol, acetone or methanol and then freed of organic solvent residues with water.

16: The process according to claim 15, wherein the solvent used to wash the crystallized 2,6-bis(methionyl)-1,4-diketopiperazine is at least partly recycled.

17: A process for isolating 2,6-bis(methionyl)-1,4-diketopiperazine from a reaction mixture, comprising:
A1) diluting the reaction mixture comprising 2,6-bis(methionyl)-1,4-diketopiperazine with a polar solvent and water, with crystallization of 2,6-bis(methionyl)-1,4-diketopiperazine; or
A2) diluting the reaction mixture comprising 2,6-bis(methionyl)-1,4-diketopiperazine only with water without prior dilution with an organic solvent with or without pressure retention, with crystallization of 2,6-bis(methionyl)-1,4-diketopiperazine; or
A3) cooling the reaction mixture down or allowing it to cool down without addition of substances until a temperature, of 135.0° C. to less than 145.0° C. is reached, at which the diketopiperazine starts to crystallize, and finally diluting it and hence cooling it by addition of water, with crystallization of 2,6-bis(methionyl)-1,4-diketopiperazine;
B) removing the crystallized 2,6-bis(methionyl)-1,4-diketopiperazine obtained in A1) or A2) or A3) in solid form and washing it with a solvent, and finally washing it with water.

18: A process for preparing 2,6-bis(methionyl)-1,4-diketopiperazine of formula (I): 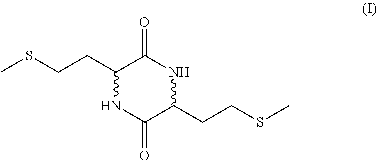
comprising:
a) heating a reaction mixture comprising methionine and a polar protic solvent at a temperature of less than 170.0° C., while passing an inert gas stream over or through the reaction mixture, in order to remove the water formed in the reaction from the reaction mixture;
b) obtaining 2,6-bis(methionyl)-1,4-diketopiperazine of formula (I);
c1) diluting the reaction mixture with polar solvent and water to crystallize 2,6-bis(methionyl)-1,4-diketopiperazine; or
c2) diluting the reaction mixture only with water without prior dilution with an organic solvent with or without pressure retention, to crystallize 2,6-bis(methionyl)-1,4-diketopiperazine; or
c3) cooling the reaction mixture down or allowing it to cool down without addition of substances until a temperature of 135.0° C. to less than 145.0° C., is reached, at which the diketopiperazine starts to crystallize, and finally diluting it and hence cooling it by addition of water, to crystallize 2,6-bis(methionyl)-1,4-diketopiperazine.
